(19) United States
(12) Patent Application Publication
(10) Pub. No.: US 2021/0069143 A1
(43) Pub. Date: Mar. 11, 2021

(54) LOW-ALCOHOL ANTIMICROBIAL COMPOSITION AND USE THEREOF

(71) Applicant: Zurex Pharma, Inc., Middleton, WI (US)

(72) Inventor: Carmine J. Durham, Middleton, WI (US)

(21) Appl. No.: 16/618,779

(22) PCT Filed: Jun. 1, 2018
(86) PCT No.: PCT/US18/35720

§ 371 Date: Dec. 2, 2019

Related U.S. Application Data
(60) Provisional application No. 62/514,635, filed on Jun. 2, 2017.

(51) Int. Cl. A61K 31/216; A61K 31/045; A61K 47/12; A61K 47/10; A61K 47/46; A61K 47/18; A61K 47/36; A61P 31/02

(52) U.S. Cl. CPC A61K 31/216; A61K 31/045; A61K 47/12; A61P 31/02; A61K 47/46; A61K 47/18; A61K 47/36; A61K 47/10

(57) ABSTRACT

Low-alcohol antimicrobial compositions and gamma-sterilized or gamma-sterilizable antimicrobial compositions comprising alcohol and parabens, and their use as a topical antimicrobial agent.

### BACKGROUND OF THE INVENTION

Topical antimicrobials are used in a variety of applications to prevent infection and the spread of bacteria and viruses. However, the use of conventional antibiotic compounds in such compositions has increased the prevalence of antibiotic resistant strains of bacteria. High-concentration alcohol compositions can be effective, but they are harsh on skin and typically are flammable, making storage and use dangerous in some contexts.

Antimicrobial compositions also can become contaminated during manufacture, despite their antimicrobial activity. However, many antimicrobial products have components that degrade under sterilizing conditions such as heat/pressure or gamma radiation. While sterile filling can be used in some situations, the process adds considerable expense to manufacturing.

Thus, there remains a need for new topical antimicrobial compositions.

### BRIEF SUMMARY OF THE INVENTION

Provided herein is a low-alcohol antimicrobial composition, which comprises about 18% or less of an alcohol, and alkyl para-hydroxybenzoate (paraben).

In another aspect, there is provided a sterile or sterilizable antimicrobial composition comprising alcohol and paraben.

Also provided herein is a method of disinfecting skin or an open soft-tissue wound by applying the low-alcohol antimicrobial composition to the skin or open soft-tissue wound to be disinfected, and a method of treating or protecting a mammalian teat by applying the low-alcohol antimicrobial composition to the teat.

Related compositions and methods also are provided as is apparent from the following detailed description.

### DETAILED DESCRIPTION OF THE INVENTION

Provided herein are antimicrobial compositions comprising a combination of alcohol and one or more paraben that provide unique and advantageous properties, particularly for use as topical antimicrobials, such as hand wash compositions and patient surgical skin preparation (surgical prep) compositions. These formulations provide good antimicrobial efficacy without requiring high levels of alcohol (allowing for low-flammability compositions) and without requiring components that breakdown under sterilizing autoclave or, particularly, gamma irradiation, thereby allowing for sterile or sterilizable compositions.

The compositions comprise alcohol and one or more parabens in the amounts set forth herein, and can comprise other components and excipients as described herein. However, in some embodiments the compositions are free or substantially free of one or more (or all) such other components or excipients. In this respect, substantially free means that these components or excipients are present in an amount that provides no observable or detectable effect on the composition as compared to the same composition without the component or excipient. In the case of a biologically active component, such as another antimicrobial component, substantially free means that the amount of the component is so small as to have no observable or detectable biological effect (e.g., no antimicrobial effect). Completely free means no amount or no detectable amount.

According to one aspect, the disclosure provides a low-alcohol antimicrobial composition, which comprises about 18 wt. % or less of an alcohol and one or more an alkyl para-hydroxybenzoates (i.e., “parabens”). The low-alcohol formulation allows for a low-flammability composition that nevertheless retains good antimicrobial efficacy.

Any alcohol suitable for antimicrobial use on skin can be used. In most cases, the alcohol will be a C1-C6, C1-C4, or C1-C3 alcohol (e.g., methanol, ethanol, n-propanol or isopropanol). The alcohol can be present any amount less than about 18% based on the weight of the total composition. In some embodiments, the composition comprises about 15 wt. % or less alcohol (e.g., about 14 wt. % or less, about 13 wt. % or less, about 12 wt. % or less, or about 11 wt. % or less) or about 10 wt. % or less (e.g., about 9 wt. % or less, about 8 wt. % or less, about 7 wt. % or less, or about 6 wt. % or less). In other embodiments, the composition comprises about 5 wt. % or less alcohol (e.g., about 4 wt. % or less, about 3 wt. % or less, about 2 wt. % or less, or even about 1 wt. % or less). In conjunction with the foregoing upper limits, the composition will typically comprise about 0.1 wt. % or more alcohol (e.g., about 0.5 wt. % or more, about 1 wt. % or more, about 1.5 wt. % or more, about 2 wt. % or more alcohol, or even about 2.5% or more or about 3% or more alcohol). The composition can comprise a mixture of alcohols (e.g., a mixture of C1-C6 alcohols, C1-C4 alcohols, or C1-C3 alcohols), in which case the foregoing upper and lower limits apply to the total amount of C1-C6 alcohols in the composition. As used herein, the phrase “at least” a given amount “X” is intended to be equivalent to (and interchangeable with) the phrase “X” amount “or more.” S

In some embodiments, the compositions provided herein are formulated to have a relatively high flash point, such that they are substantially non-flammable and safe for storage and use in a variety of contexts where flammable materials are not safe. In one embodiment, the composition has a flash point of about 50° C. or greater, such as about 60° C. or greater, when tested under ASTM D3278.

Any suitable alkyl para-hydroxybenzoate (paraben) can be used in the composition. Suitable alkyl para-hydroxybenzoates include methyl-, ethyl-, propyl-, and butyl-para-hydroxybenzoate, and combinations thereof. There is no particular limit on the amount of paraben used, and it can be used up to the solubility limit of the particular paraben paraben used. In some embodiments, the composition comprises about 10 mM or more alkyl para-hydroxybenzoate, such as about 12 mM or more, about 15 mM or more, about 20 mM or more, or even about 25 mM or more or 30 mM or more. In some cases, the composition will comprise no more than about 90 mM, no more than about 60 mM, or no more than about 50 mM alkyl para-hydroxybenzoate, or even about 40 mM or less alkyl para-hydroxybenzoate. Any of the foregoing amounts also can be expressed as ranges (e.g., about 10-90, about 10-60 mM, about 10-50 mM, about 10-40 mM; about 12-90 mM, about 12-60 mM, about 12-50 mM, about 12-40 mM, about 15-90 mM, about 15-60 mM, about 15-50 mM, about 15-40 mM, about 20-90 mM, about 20-60 mM, about 20-50 mM, about 20-40 mM, about 25-90 mM, about 25-60 mM, about 25-50 mM, about 25-40 mM, about 30-90 mM, about 30-60 mM, about 30-50 mM, about 30-40 mM. Expressed as weight percentages, the composition can in some embodiments comprise about 0.1 wt. % or more paraben, such as about 0.2 wt. % or more, about 0.3 wt. % or more, about 0.4 wt. % or more, or about 0.5 wt. % or more. Generally, the paraben will constitute about 2 wt. % or less of the composition, such as about 1.5 wt. % or less, or even about 1 wt. % or less. In some embodiments, the amount of methyl paraben, ethyl paraben, or combination thereof will be about 0.8 wt. % or less, such as about 0.4 wt. % or less (e.g., about 0.1% to about 0.8% or about 0.1% to about 0.4%). In other embodiments, the amount of butyl paraben, propyl paraben, or combination thereof will be about 0.2 wt. % or less or even 0.19% or less (e.g., about 0.01 to about 0.2% or about 0.01 to 0.19%.) Any of the foregoing amounts may also be expressed as ranges (e.g., about 0.1-2 wt. %, about 0.1-1.5 wt. %, about 0.1-0.8 wt. %, about 0.1-0.4 wt. %, about 0.2-2 wt. %, about 0.2-1.5 wt. %, about 0.2-1 wt. %,about 0.2-0.8 wt. %, about 0.2-0.4 wt. %, about 0.3-2 wt. %, about 0.3-1.5 wt. %, about 0.3-1 wt. %, about 0.3-0.8 wt. %, about 0.3-0.4 wt. %, about 0.4-2 wt. %, about 0.4-1.5 wt. %, about 0.4-1 wt. %, about 0.4-0.8 wt. %, about 0.5-2 wt. %, about 0.5-1.5 wt. %, about 0.5-1 wt. %,or about 0.5-0.8 wt. %).

The composition can comprise more than one type of alkyl para-hydroxybenzoate. For example, the composition can comprise methyl- and propyl-para-hydroxybenzoate. When more than one type of alkyl para-hydroxybenzoate is used, the combined amount is generally within the ranges discussed herein. In one embodiment, the composition comprises about 0.05-0.5 wt. % or about 0.05-0.4 wt. % (e.g., 0.05-0.3 wt. % or 0.1-0.2 wt. %) of propyl-parahydroxybenzoate, and the remaining portion of the alkyl para-hydroxybenzoate is methyl-para-hydroxybenzoate (e.g., about 0.2-0.6 wt. %, about 0.3-0.5 wt. % or about 0.3-0.4 wt. %). The alkyl para-hydroxybenzoate can be supplied by any source, for instance, a salt of an alkyl para-hydroxybenzoate or an alkyl para-hydroxybenzoic acid.

In some embodiments, the composition also comprises an organic acid or its conjugate base, such as a carboxylic acid or its conjugate base (e.g., a C1-C8 or C2-C6 carboxylic acid). Examples of organic acids with at least one carboxylic acid functional group include carboxylic acid, formic acid, acetic acid, stearic acid, lactic acid, madelic acid, acrylic acid, oleic acid, benzoic acid, citric acid, salicylic acid, tartaric acid, succinic acid, pthalic acid, malonic acid, methacrylic acid, oxalic acid, ispcitric acid, crotonic acid, glyceric acid, p-Toluic acid, propanoic acid, heptanoic acid, butanoic acid, tartronic acid, nitroacetic acid, cyanoecetic acid, methoxyacetic acid, flouroacetic acid, chloroacetic acid, bromoacetic acid, dichloroacetic acid, glutaric acid, trichloroacetic acid, malic acid, hexanoic acid, trimellitic acid, trimesic acid, aconitic acid, tricarballylic acid and gallic acid. In one embodiment, the organic acid is acetic acid, lactic acid, proprionic, fumaric acid, or citric acid. In another embodiment, the organic acid includes three carboxylic acid functional groups. Examples of organic acids with three carboxylic acid groups include citric acid, isocitric acid, trimellitic acid, trimesic acid, tricarballylic acid, aconitic acid and mixtures thereof. The conjugate bases of the foregoing acids also are included. In a particular embodiment, the organic acid or conjugate base is citric acid or citrate.

The composition can comprise any suitable amount of the organic acid or conjugate base, particularly citric acid or citrate, up to the solubility limit. For instance, the composition can comprise about 0.1 M or more, such as about 0.2 M or more, or about 0.3 M or more of an organic acid or conjugate base. Typically, the composition will comprise about 3M or less of the organic acid or conjugate base, or about 2 M or less of the organic acid or conjugate base, such as about 1 M or less, about 0.8 M or less, or about 0.5 M or less. The foregoing amounts also can be expressed as ranges (e.g., about 0.1-2 M, about 0.1-1 M, about 0.1-0.8 M, about 0.2-2 M, about 0.2-1 M, about 0.2-0.8 M, about 0.3-2 M, about 0.3-1 M, about 0.3-0.8 M, about 0.3-0.5 M). Any sub-range thereof also is contemplated.

The organic acid or its conjugate base, particularly citric acid or citrate, can be provided by any suitable source. For instance citrate can be provided by citric acid, a citrate salt, or a combination thereof. Suitable salts include sodium, potassium, magnesium, or calcium citrate salts. Furthermore, the citrate salt can be a monvalent salt or a multivalent salt, such as a monobasic, dibasic, or tribasic citrate salt (e.g. mono-, di-, or tri-sodium citrate or mono-, di-, or tri-potassium citrate). Expressed as a weight percentage, the composition can comprise, for instance, about 1 to about 50% or about 1 to about 15 wt. % (e.g., about 2-7 wt. % or 3-5 wt. %) of an organic acid or its salt (e.g., citric acid and/or citrate salt). In one embodiment, the composition comprises about 2-7 wt. % or 3-5 wt. % citric acid and about 0.1-1 wt. % or 0.1-0.5 wt. % of a citrate salt (e.g., tribasic citrate salt).

In some embodiments, the composition is substantially or completely free of any one or more of the foregoing organic acids, bases, or salts.

In some embodiments, the composition comprises sodium, potassium, magnesium, calcium ions, or a combination thereof. The ions can be present in a concentration of about 0.1 M or more, such as 0.2 M or more, or even 0.3 M or more. The sodium, potassium, magnesium, or calcium ions can be provided by any suitable source, for instance, by use of sodium, potassium, magnesium, or calcium citrate salts as a source for the citrate. In some embodiments, the composition is substantially or completely free of one or more (or all) of the foregoing ions.

The composition can have any suitable pH depending upon the desired application. In some embodiments, the composition has a pH of about 2-8 (e.g., about 2-7, about 2-6, about 2-5, about 3-8, about 3-7, about 3-6, about 3-5, about 4-8, about 4-7, about 4-6, about 4-5, about 5-8, about 5-7 or about 5-6). The pH of the composition can be adjusted as needed using any common pH adjusting agent, typically a strong acid or base (e.g., HC1 or NaOH).

The composition can further comprise an emollient. Suitable emollients include, for instance, glycerol, propylene glycol, lanolin, glycerin, sorbitol, D-panthenol, poly ethylene glycol (PEG) (e.g., mw. 200-10,000) and esters thereof, acyl lactylates, polyquaternium compounds (polyquaternium-7), glycerol cocoate/laurate, PEG-7 glycerol cocoate, stearic acid, hydrolyzed silk peptide, silk protein, aloe vera gel, guar hydroxypropyltrimonium chloride, alkyl poly glucoside/glyceryl luarate, shea butter and coco butter. In some embodiments, emollients will typically be present in an amount of about 5 wt. % or more, such as about 10 wt. % or more, or even about 15 wt. % or more. In some embodiments, the composition will generally have no more than about 50 wt. % emollients, such as about 40 wt. % or less, about 30 wt. % or less, or about 25 wt. % or less. In other embodiments (e.g., cold-weather formulations), more emollient may be used, such as about 20 wt. % or more, about 30 wt. % or more, about 40 wt. % or more, about 50 wt. % or more, or even about 60 wt. % or more or even about 75 wt. % or more. In one embodiment, the composition comprises about 10-30% propylene glycol (e.g., about 15-25 wt. % propylene glycol). In some embodiments, the composition is substantially or completely free of emollients.

In some embodiments, the composition can further comprise a barrier or film-forming agent, or a thickener. Suitable barrier and film-forming agents and thickeners include, for instance, polyvinylpyrrolidone (PVP), polyvinylalcohol (PVA or PVOH), polyacrylate, polyacrylamide, latex, carbomer, glycerol, hemicelluloses (e.g., arabinoxylanes and glucomannanes); plant gum materials (e.g., guar gum, gum arabic, and johannistree gums); cellulose and derivatives thereof (e.g., methyl cellulose, ethyl cellulose, hydroxypropyl cellulose, hydroxyethyl cellulose, carboxymethyl cellulose, carboxyethyl cellulose; methylhydroxypropylcellulose (HPMC) and ethylhydroxyethylcellulose); starch and starch derivatives (e.g., hydroxyethyl starch or cross linked starch); microbial and sea weed polysaccharides (e.g., xanthan gum, sodium alginate, carrageenan, curdlan, pullulan, and dextran), dextran sulfate, whey, collagen, pectin, gelatin, chitosan, chitosan derivatives, and polysulfonic acids and their salts. Clays and modified clays (e.g., bentonite or laponite), colloidal alumina or silica, and fatty acids or salts thereof can also be used as thickeners, co-thickeners, or stability agents for thickeners. The amount used will depend upon the particular agent selected. Generally, when used, the barrier or film forming agent, or thickener, will be present in an amount of about 0.1 wt. % or more, such as about 1 wt. % or more, 2 wt. % or more, 5 wt. % or more, or even 10 wt. % or more. Typically, the barrier or film forming agent, or thickener, constitute no more than about 40 wt. % of the composition, such as about 35 wt. % or less, 30 wt. % or less, or 25 wt. % or less. In one embodiment, the composition comprises xantham gum in an amount of about 0.1-5 wt. % (e.g., about 0.5-3 wt. %). In some embodiments, the composition is substantially or completely free of a barrier or film-forming agent, and/or thickener.

In some embodiments, the composition comprises at least one gelling agent, which can be the same as, or different from, the barrier or film-forming agent or thickener. Gelling agents include any of those agents described as above with respect to the barrier or film-forming agents and thickeners that can produce a gel. By way of non-limiting examples, the gelling agent can be polyvinylpyrrolidone (PVP), polyvinylalcohol (PVA or PVOH), polyacrylate (e.g., cross-linked polyacrylic acid polymers such as the CARBOPOL® products by Lubrizol Corp., or acrylate copolymer Capigel™ by Seppic, Inc.), polyacrylamide, latex, carbomer, cellulose or derivative thereof (e.g., methyl cellulose, ethyl cellulose, hydroxypropyl cellulose, hydroxyethyl cellulose, carboxymethyl cellulose, carboxyethyl cellulose; methylhydroxypropylcellulose (HPMC) and ethylhydroxyethylcellulose). The amount used will depend upon the particular agent selected. Generally, when used, the gelling agent will generally be present in an amount of about 0.1 wt % or more, such as about 1 wt. % or more, about 2 wt. % or more, or even about 5 wt. % or more. Typically, the gelling agent will constitute no more than about50 wt. % or no more than about 40 wt. % of the composition, such as about 35 wt. % or less, or 30 wt. % or less. By way of further illustration, in some applications, the composition will comprise about 1-50 wt. %, or about 1-20 wt. %, such as about 2-10 wt. % or even about 2-5 wt. %, of the gelling agent. In other applications, the composition can comprise about 10-30 wt. % or 20-30 wt. % gelling agent. In some embodiments, the composition is substantially or completely free of gelling agents.

In some embodiments, the composition can further comprise a surfactant or foaming agent in any suitable amount (e.g., about 0.1-40 wt. %, such as about 1-20 wt. % or about 1-5 wt. %). Surfactants include anionic, cationic, nonionic, zwitterionic and amphoteric surfactants, and can be high foaming, low foaming, moderate foaming, or non foaming type surfactants. Anionic surfactants include, for example, linear alkyl benzene sulfonic acid,linear alkyl benzene sulfonate, alkyl sulfomethyl ester, α-olefin sulfonate, alcohol ether sulfate, alkyl sulfate, alkylsulfo- and dialkylsulfo succinate, and salts thereof. Nonionic surfactants include, for example, alkyl polyglucoside, alkyl ethoxylated alcohol, alkyl propoxylated alcohol, ethoxylatedpropoxylated alcohol, alkylphenol ethoxylates, sorbitan, sorbitan ester, alkanol amide, and polyethoxylated polyoxypropylene block copolymers (poloxamers). Amphoteric surfactants include, for example, alkyl betaines and alkyl amphoacetates (e.g., cocoamidopropyl betaine, sodium cocoamphoacetate, sodium lauroamphoacetate and sodium cocoamphodiacetate). In other embodiments, the composition is substantially or completely free of surfactactants (e.g., no observable foaming effect in the case of a foaming agent). In many cases, amounts of about 0.05 wt. % or about 0.01 wt. % or less of surfactant will be considered substantially free.

The composition can further include a colorant, such as a food-grade colorantln other embodiments, the composition is free of colorant.

The principal carrier for the topical composition will typically be water. Generally, the composition will comprise water in an amount of about 15 wt. % or more, about 30 wt. % or more, or about 40 wt. % or more, such as about 60 wt. % or more, or even 70 wt. % or more (e.g., 80 wt. % or more).

The composition can further comprise additional antibiotic or antimicrobial agents, particularly topical antibiotic or antimicrobial agents, such as such as iodine-containing antiseptics (e.g., iodine or iodophors); chlorine based antiseptics (e.g., hypochlorites (e.g., sodium hypochlorite; anolyte); antiseptic plant oils; phenols; quaternary ammonium compounds; antiseptic surfactants; bisbiguanides (e.g., chorhexidine); terpenes; sodium bicarbonate; sulfates; guanidine salts; formaldehyde-releasing compounds; ascorbic acid; benzyl alcohols; trihalocarbanilides; phenolic compounds; macrocyclic antibiotic or antifungals; and peracids/peroxides.

While the compositions described herein can be formulated with additional antibiotic or antiseptic components, one of the advantages of at least some compositions described herein is that such additional antibiotic or antiseptic agents are not required. Thus, in additional embodiments, the compositions described herein can be substantially free (e.g., contains less than an antimicrobial-effective amount) or completely free of one or more common topical antibiotic or antiseptic agents, such as those mentioned above. Alternatively, the compositions can be substantially free or completely free of any antiseptic or antimicrobial agent other than the combination of alkyl para-hydroxybenzoate and alcohol. In some embodiements, the composition is substantially or completely free of redox compounds, particularly redox indicator dyes, such as methylene blue. In many cases, amounts below about 0.01 wt. % will be considered substantially free, but the ultimate determination may depend upon the particular component in question.

In some embodiments, the composition comprises an active (antimicrobial) ingredient that consists of (a) alcohol, (b) a paraben, and optionally (c) citric acid or citrate. In some embodiments, the composition consists essentially of, or consists of, the alcohol, alkyl para-hydroxybenzoate, and, optionally, citric acid, emollient, thickener, urea, and pH adjusting agent, as described above. A composition that is believed to be particularly advantageous comprises, consists essentially of, or consists of (a) about 2 wt. % to about 7 wt. % or about 2 wt. % to about 5 wt % of a C1-C3 alcohol; (b) about 0.4 wt. % to the solubility limit, or about 0.4 to about 1 wt. % alkyl para-hydroxybenzoate (e.g., about 0.1-0.3 wt. % propylparaben and about 0.3-0.7 wt % methyl paraben); and (d) about 1-7 wt. % citric acid (e.g., about 3-6 wt. %) and, optionally, citrate salt (about 0.2 wt % to about 1.0 wt. %). In some embodiments, the composition may optionally further comprise one or more of propylene glycol, glycerin, lanolin, urea, or xanthan gum (e.g., about 10-30 wt. % propylene glycol, glycerin, urea, or combination thereof; about 1-5 wt. % urea; and/or about 0.2-5 wt. % xantham gum. The composition will generally comprise a balance of a suitable solvent (e.g., water), and optional pH adjusting agent as necessary to provide a suitable pH (e.g., a pH of about 3-5).

In another aspect, the disclosure provides a sterile or sterilizable antimicrobial composition comprising alcohol and paraben, which is substantially or completely free of any component that degrades upon application of a sterilizing dose of gamma radiation. In another embodiment, the composition is substantially or completely free of components that degrade upon applicaton of sterilizing autoclave conditions.

A composition is considered to be sterile if it has been subjected to the minimum sterilisation conditions (e.g., a sterilization dose) sufficient to provide a sterility assurance level (SAL) of at least 10⁻³, 10⁻⁴, 10⁻⁵, 10⁻⁶, 10⁻⁷, or 10⁻⁸. Any sterilization method can be employed.

Methods of autoclaving liquids to achieve the desired SAL are known in the art, any of which can be employed to provide the sterile composition.

In particular embodiments, the composition is gamma-radiation sterlized or gamma-radiation sterilizable (i.e., sterilized by gamma irradiation). Gamma radiation can be applied by any technique known in the art. The primary industrial source of gamma rays are radionucleotide elements, such as Cobalt 60, but any source can be used. A sterilizing dose of radiation can be established for a given application by known methods. Standards for validation of gamma sterilization are available (e.g., ANSI/AAMI ST67, AAMI TIR 33, and ANSI/AAMI/ISO 11137). Generally, the dose should be sufficient to provide a sterility assurance level (SAL) of at least 10⁻³, 10⁻⁴, 10⁻⁵, 10⁻⁶, 10⁻⁷, or 10⁻⁸. In some embodiments, the sterilizing dose of radiation is about 15 kGy or more, about 17.5 kGy or more, about 20 kGy or more, about 22.5 kGy or more, about 25 kGy or more, about 27.5 kGy or more, about 30 kGy or more, about 32.5 kGy or more, or about 35 kGy or more (e.g., about 40 kGy or more).

Any alcohol suitable for antimicrobial use on skin can be used. In most cases, the alcohol will be a C1-C6 or C1-C3 alcohol (e.g., methanol, ethanol, n-propanol or isopropanol). In some embodiments, the formulation can comprise relatively low amounts of alcohol. Thus, for instance, the alcohol can be present an amount less than about 18% based on the weight of the total composition. In some embodiments, the composition comprises about 15 wt. % or less alcolol (e.g., about 14 wt. % or less, about 13 wt. % or less, about 12 wt. % or less, or about 11 wt. % or less) or about 10 wt. % or less (e.g., about 9 wt. % or less, about 8 wt. % or less, about 7 wt. % or less, or about 6 wt. % or less). In other embodiments, the composition comprises about 5 wt. % or less alcohol (e.g., about 4 wt. % or less, about 3 wt. % or less, about 2 wt. % or less, or even about 1 wt. % or less). In some emobodiments, the compositions provided herein are formulated to have a relatively high flash point, such that they are substantially non-flammable and safe for storage and use in a variety of contexts where flammable materials are not safe. In one embodiment, the composition has a flash point of about 50° C. or greater, such as about 60° C. or greater, when tested under ASTM D3278.

However, in other embodiments of the sterile or sterilizable composition, higher levels of alcohol can be used. For example, the composition can comprise up to about 80% alcohol, such as about 75% or less alcohol, about 70% or less alcohol, about 65% or less alcohol, about 60% or less alcohol, or about 55% or less alcohol (e.g., about 50% or less, about 45% or less, about 40% or less, about 35% or less, or even about 30% or less alcohol).

In conjunction with the foregoing upper limits, the composition will typically comprise about 0.1 wt. % or more alcohol (e.g., about 0.5 wt. % or more, about 1 wt. % or more, about 1.5 wt. % or more, about 2 wt. % or more alcohol, or even about 2.5% or more or about 3% or more alcohol). In some embodiments, the composition comprises about 5% or more alcohol, or about 10% or more alcohol, such as about 15% or more alcohol. In compositions with higher amounts of alcohol, the composition can comprise about 20% or more alcohol (e.g., about 25%, or more, about 30% or more, or about 35% or more alcohol). The composition can comprise a single type of alcohol, or a mixture of alcohols (e.g., a mixture of C1-C6 alcohols or C1-C4 alcohols), in which case the foregoing upper and lower limits apply to the total amount of C1-C6 alcohols in the composition.

Any suitable alkyl para-hydroxybenzoate (paraben) can be used in the composition. Suitable alkyl para-hydroxybenzoates include methyl-, ethyl-, propyl-, and butyl-para-hydroxybenzoate, and combinations thereof. There is no particular limit on the amount of paraben used, and it can be used up to the solubility limit of the particular paraben paraben used. In some embodiments, the composition comprises about 10 mM or more alkyl para-hydroxybenzoate, such as about 12 mM or more, about 15 mM or more, about 20 mM or more, or even about 25 mM or more or 30 mM or more. In some cases, the composition will comprise no more than about 90 mM, no more than about 60 mM, or no more than about 50 mM alkyl para-hydroxybenzoate, or even about 40 mM or less alkyl para-hydroxybenzoate. Any of the foregoing amounts also can be expressed as ranges (e.g., about 10-90, about 10-60 mM, about 10-50 mM, about 10-40 mM; about 12-90 mM, about 12-60 mM, about 12-50 mM, about 12-40 mM, about 15-90 mM, about 15-60 mM, about 15-50 mM, about 15-40 mM, about 20-90 mM, about 20-60 mM, about 20-50 mM, about 20-40 mM, about 25-90 mM, about 25-60 mM, about 25-50 mM, about 25-40 mM, about 30-90 mM, about 30-60 mM, about 30-50 mM, about 30-40 mM. Expressed as weight percentages, the composition can in some embodiments comprise about 0.1 wt. % or more paraben, such as about 0.2 wt. % or more, about 0.3 wt. % or more, about 0.4 wt. % or more, or about 0.5 wt. % or more. Generally, the paraben will constitute about 2 wt. % or less of the composition, such as about 1.5 wt. % or less, or even about 1 wt. % or less. In some embodiments, the amount of methyl paraben, ethyl paraben, or combination thereof will be about 0.8 wt. % or less, such as about 0.4 wt. % or less (e.g., about 0.1% to about 0.8% or about 0.1% to about 0.4%). In other embodiments, the amount of butyl paraben, propyl paraben, or combination thereof will be about 0.2 wt % or less or even 0.19% or less (e.g., about 0.01 to about 0.2% or about 0.01 to 0.19%.) Any of the foregoing amounts may also be expressed as ranges (e.g., about 0.1-2 wt. %, about 0.1-1.5 wt. %, about 0.1-0.8 wt. %, about 0.1-0.4 wt %, about 0.2-2 wt. %, about 0.2-1.5 wt. %, about 0.2-1 wt. %,about 0.2-0.8 wt. %, about 0.2-0.4 wt %, about 0.3-2 wt. %, about 0.3-1.5 wt. %, about 0.3-1 wt. %, about 0.3-0.8 wt. %, about 0.3-0.4 wt %, about 0.4-2 wt. %, about 0.4-1.5 wt. %, about 0.4-1 wt. %,cabout 0.4-0.8 wt. %, about 0.5-2 wt. %, about 0.5-1.5 wt. %, about 0.5-1 wt. %,or about 0.5-0.8 wt. %).

The composition can comprise more than one type of alkyl para-hydroxybenzoate. For example, the composition can comprise methyl- and propyl-para-hydroxybenzoate. When more than one type of alkyl para-hydroxybenzoate is used, the combined amount is generally within the ranges discussed herein. In one embodiment, the composition comprises about 0.05-0.5 wt % or about 0.05-0.4 wt. % (e.g., 0.05-0.3 wt % or 0.1-0.2 wt %) of propyl-parahydroxybenzoate, and the remaining portion of the alkyl para-hydroxybenzoate is methyl-para-hydroxybenzoate (e.g., about 0.2-0.6 wt. %, about 0.3-0.5 wt. %, or about 0.3 to 0.4 wt. %). The alkyl para-hydroxybenzoate can be supplied by any source, for instance, a salt of an alkyl para-hydroxybenzoate or an alkyl parahydroxybenzoic acid.

In some embodiments, the composition also comprises an organic acid or its conjugate base, such as a carboxylic acid or its conjugate base (e.g., a C1-C8 or C2-C6 carboxylic acid). Examples of organic acids with at least one carboxylic acid functional group include carboxylic acid, formic acid, acetic acid, stearic acid, lactic acid, madelic acid, acrylic acid, oleic acid, benzoic acid, citric acid, salicylic acid, tartaric acid, succinic acid, pthalic acid, malonic acid, methacrylic acid, oxalic acid, ispcitric acid, crotonic acid, glyceric acid, p-Toluic acid, propanoic acid, heptanoic acid, butanoic acid, tartronic acid, nitroacetic acid, cyanoecetic acid, methoxyacetic acid, flouroacetic acid, chloroacetic acid, bromoacetic acid, dichloroacetic acid, glutaric acid, trichloroacetic acid, malic acid, hexanoic acid, trimellitic acid, trimesic acid, aconitic acid, tricarballylic acid and gallic acid. In one embodiment, the organic acid is acetic acid, lactic acid, proprionic, fumaric acid, or citric acid. In another embodiment, the organic acid includes three carboxylic acid functional groups. Examples of organic acids with three carboxylic acid groups include citric acid, isocitric acid, trimellitic acid, trimesic acid, tricarballylic acid, aconitic acid and mixtures thereof. The conjugate bases of the foregoing acids also are included. In a particular embodiment, the organic acid or conjugate base is citric acid or citrate.

The composition can comprise any suitable amount of the organic acid or conjugate base, particularly citric acid or citrate, up to the solubility limit. For instance, the composition can comprise about 0.1 M or more, such as about 0.2 M or more, or about 0.3 M or more of an organic acid or conjugate base. Typically, the composition will comprise about 3M or less of the organic acid or conjugate base, or about 2 M or less of the organic acid or conjugate base, such as about 1 M or less, about 0.8 M or less, or about 0.5 M or less. The foregoing amounts also can be expressed as ranges (e.g., about 0.1-2 M, about 0.1-1 M, about 0.1-0.8 M, about 0.2-2 M, about 0.2-1 M, about 0.2-0.8 M, about 0.3-2 M, about 0.3-1 M, about 0.3-0.8 M, about 0.3-0.5 M). Any sub-range thereof also is contemplated.

The organic acid or its conjugate base, particularly citric acid or citrate, can be provided by any suitable source. For instance citrate can be provided by citric acid, a citrate salt, or a combination thereof. Suitable salts include sodium, potassium, magnesium, or calcium citrate salts. Furthermore, the citrate salt can be a monvalent salt or a multivalent salt, such as a monobasic, dibasic, or tribasic citrate salt (e.g. mono-, di-, or tri-sodium citrate or mono-, di-, or tri-potassium citrate). Expressed as a weight percentage, the composition can comprise, for instance, about 1 to about 50% or about 1 to about 15 wt. % (e.g., about 2-7 wt. % or 3-5 wt. %) of an organic acid or its salt (e.g., citric acid and/or citrate salt). In one embodiment, the composition comprises about 2-7 wt. % or 3-5 wt. % citric acid and about 0.1-1 wt. % or 0.1-0.5 wt. % of a citrate salt (e.g., tribasic citrate salt).

In some embodiments, the composition is substantially or completely free of any one or more of the foregoing organic acids, bases, or salts.

In some embodiments, the composition comprises sodium, potassium, magnesium, calcium ions, or a combination thereof. The ions can be present in a concentration of about 0.1 M or more, such as 0.2 M or more, or even 0.3 M or more. The sodium, potassium, magnesium, or calcium ions can be provided by any suitable source, for instance, by use of sodium, potassium, magnesium, or calcium citrate salts as a source for the citrate. In some embodiments, the composition is substantially or completely free of one or more (or all) of the foregoing ions.

The composition can have any suitable pH depending upon the desired application. In some embodiments, the composition has a pH of about 2-8 (e.g., about 2-7, about 2-6, about 2-5, about 3-8, about 3-7, about 3-6, about 3-5, about 4-8, about 4-7, about 4-6, about 4-5, about 5-8, about 5-7 or about 5-6). The pH of the composition can be adjusted as needed using any common pH adjusting agent, typically a strong acid or base (e.g., HC1 or NaOH).

The composition can further include a colorant, such as a food-grade colorant, that does not degrade under the sterilizing gamma radiation or sterilizing autoclave (heat/pressure) conditions. In other embodiments, the composition is free of colorant.

The sterile or sterilizable composition can further comprise other components typically contained in a topical antimicrobial composition, particular a hand wash or patient surgical prep composition, provided the components do not degrade under the sterilizing gamma radiation or sterilizing autoclave (heat/pressure) conditions. However, one advantage of the sterile or sterilizable composition is that no other components are required to provide a serviceable composition with excellent antimicrobial effect besides the alcohol and paraben.

Thus, for instance, the composition can further comprise an emollient, a barrier or film-forming agent, a thickener, a gelling agent, a surfactant or foaming agent, or additional antibiotic or antimicrobial agents (other than the alcohol and paraben), as described with respect to the low-alcohol composition provided herein, provide such components do not degrade under sterilizing gamma radiation or sterilizing autoclave (heat/pressure) conditions. However, in some embodiments, the sterile or sterilizable composition is substantially or completely free of an emollient; is substantially or completely free of a barrier or film-forming agent; is substantially or completely free of a thickener; is substantially or completely free of a gelling agent; is substantially or completely free of a surfactant or foaming agent; and/or is substantially or completely free of or additional antibiotic or antimicrobial agents.

In some embodiments, the sterile or sterilizable composition comprises an active (antimicrobial) ingredient that consists of (a) alcohol, (b) paraben, and optionally (c) citric acid or citrate; or an active ingredient that consists of (a) alcohol and (b) paraben. In another embodiment, the sterile or sterilizable composition consists essentially of or consists of (a) alcohol, (b) paraben, (c) water, optionally (d) citrate, and optionally (e) a food-grade colorant. In still another embodiment, the sterile or sterilizable composition consists essentially of or consists of (a) alcohol, (b) paraben, (c) water, and optionally (d) citrate; or the sterile or sterilizable composition consists essentially of or consists of (a) alcohol, (b) paraben, (c) water, and optionally (d) a food-grade colorant; or the sterile or sterilizable composition consists essentially of or consists of (a) alcohol, (b) paraben, and (c) water. In any of these embodiments, the amounts of the components (e.g., alcohol, paraben, citrate or citric acid) can be any as previously described. A composition that is believed to be particularly advantageous comprises, consists essentially of, or consists of (a) about 20 to about 80 wt % or about 50 to about 75 wt. % of a C1-C3 alcohol; (b) about 0.4 wt. % to the solubility limit, or about 0.4 to about 1 wt. %, of an alkyl para-hydroxybenzoate (e.g., about 0.1 to about 0.3 wt. % propylparaben and about 0.3 to about 0.7 wt. % methyl paraben); with or without (d) about 1 wt. % to about 15 wt. % or about 1 wt. % to about 7 wt. % citric acid (e.g., about 3-6 wt. % citric acid) and, optionally, citrate salt (about 0.2 wt. % to about 1.0 wt. % citrate salt); and with or without a non-redox colorant (e.g., food grade colorant).

Any of the paraben and alcohol containing antimicrobial compositions (low-alcohol and/or sterile or sterilizable compositions) provided herein can formulated as a liquid, foam, or gel, and topically applied in any manner depending upon the end use. For instance, the composition can be applied by dipping, wiping, brushing, or spraying the composition onto the skin or wound to be disinfected, or onto the skin of a teat to be treated or protected. The compositions, especially liquid compositions, can have any suitable viscosity. In some instances, it may be desired to use a thicker composition so that the composition is retained on the skin for a longer period of time without dripping. Thus, the composition can be formulated so as to have a higher viscosity, such as about 50 cP or more, about 100 cP or more, about 200 cP or more, about 500 cP or more, about 1000 cP or more, about 2500 cP or more, or even about 5000 cP or more. The viscosity of the composition will typically be less than about 10,000 cP. In other embodiments, no viscosity enhancing agents are used. Thus, for instance, the composition can have a low viscosity (e.g., about 5 cP or less, about 2 cP or less, or about 1 cP or less), or a viscosity similar to that of water or the alcohol used in the composition. Viscosity refers to the kinematic viscosity measured at standard temperature and pressure (25° C. and 1 atm).

The compositions described herein can be provided in any suitable container, such as an applicator or container configured to be introduced into an applicator. In one embodiment, the applicator can comprise an absorbent material. For instance, the applicator comprises a handle portion comprising a chamber for housing the antimicrobial composition (which can be in its own separate housing or container), and an applicator portion comprising an absorbent material in fluid communication with the handle portion. A container or fluid housing configured to be introduced into an application can, for instance, be a sealed container comprising at least one end that is openable or breachable upon insertion into the applicator (e.g., insertion into the handle portion of the applicator). One example of a suitable applicator and fluid housing is described in U.S. Pat. No. 9,844,654.

In another embodiment, the container is an applicator suitable for applying the composition to the skin of the teat of a dairy animal. Such an applicator can comprise, for instance, a cup portion of a size and shape that will allow the teat to be inserted into the cup and contact the topical composition. In yet another embodiment, the composition can be supplied in container (e.g., rigid or compressible bottle or pouch) that can be used to dispense the product directly or to supply product to a dispenser. For instance, the product can be in a compressible or rigid bottle or pouch that is inserted into a dispenser (e.g., a wall dispenser or handheld dispenser) of the type used for hand washing compositions or patient surgical prep compositions.

In still other embodiments, the composition can be absorbed into a fiber cloth or wipe. The fiber cloth or wipe can be made of any suitable material, such as any natural or synthetic polymer. Examples of suitable materials include, for instance, polyester, polypropylene, cotton, wood pulp, or rayon fibers formed into woven or non-woven sheets.

In some embodiments, the container or fiber cloth or wipe does not degrade upon application of a sterilizing dose of gamma radiation or sterilizing autoclave conditions. This is particularly advantageous when used with the sterile or sterilizable composition provided herein, as the entire composition and container can be sterilized at once. The container or fiber cloth or wipe can be sealed and packaged in a single-use package, wherein the package desirably does not degrade upon application of a sterilizing dose of gamma radiation or sterilizing autoclave conditions.

In another aspect, the disclosure provides a method of preparing a sterile antimicrobial composition as described herein, the method comprising (i) providing a sterilizable composition comprising (a) alcohol, (b) paraben, and (c) water, and (ii) applying a sterilizing dose of gamma radiation (e.g., about 15 kGy or more, about 17.5 kGy or more, about 20 kGy or more, about 22.5 kGy or more, about 25 kGy or more, about 27.5 kGy or more, about 30 kGy or more, about 32.5 kGy or more, or about 35 kGy or more) to the composition to provide a sterile antimicrobial composition. The composition used in step (i) can be any sterilizable composition described herein. Thus, in some embodiments, the sterilizable composition comprises an active (antimicrobial) ingredient that consists of (a) alcohol, (b) paraben, and optionally (c) citric acid or citrate; or an active ingredient that consists of (a) alcohol and (b) paraben. In another embodiment, the sterilizable composition consists essentially of or consists of (a) alcohol, (b) paraben, (c) water, optionally (d) citrate, and optionally (e) a food-grade colorant. In still another embodiment, the sterilizable composition consists essentially of or consists of (a) alcohol, (b) paraben, (c) water, and optionally (d) citrate; or the sterilizable composition consists essentially of or consists of (a) alcohol, (b) paraben, (c) water, and optionally (d) a food-grade colorant; or the sterilizable composition consists essentially of or consists of (a) alcohol, (b) paraben, and (c) water. In any of these embodiments, the amounts of the components are as previously described.

The sterilizing dose of radiation used should be sufficient to provide the desired SAL (e.g., at least 10⁻³, 10⁻⁴, 10⁻⁵, 10⁻⁶, 10⁻⁷, or 10⁻⁸). In some embodiments, the composition is in a container or adsorbed in a fiber cloth or wipe as described herein, optionally in packaging as described herein. All other aspects of the method are as described with respect to the other compositions and methods described herein.

In preferred embodiments, the compositions described herein provide an antimicrobial effect when applied to the skin of a mammal (e.g., a human or food-producing mammal). Without wishing to be bound by any particular theory or mechanism of action, it is believed that this effect is primarily the result of the combined (e.g., synergistic) action of the alkyl para-hydroxybenzoate and alcohol.

The methods and compositions described herein are useful with respect to the skin any type of mammal, particularly humans and food-producing mammals such as a dairy animal. For instance, the compositions can be used for wound cleansing, or pre-operative surgical site skin preparation. In this respect, there is provided herein a method for cleansing an open soft-tissue wound, or for pre-operative surgical site skin preparation, the method comprising applying the composition described herein to the wound or surgical site of the skin of the patient. The method can further comprise scrubbing the wound or skin with the composition for a suitable time, such as for about 30 seconds or more, about 60 seconds or more, about 2 minutes or more, or about 3 minutes or more.

The composition also is useful as a hand washing composition. Accordingly, the invention provides a method for washing hands comprising applying the composition described herein to the skin of the hands, optionally with scrubbing.

The compositions described herein also are useful for treating or protecting the teats of food-producing mammals susceptible to teat infections, such as mastitis, and, thus, may be particularly useful in treating or protecting the teats of dairy cows. A method of treating or protecting the teats of a food-producing mammal comprises applying the composition described herein to the skin of the teat, typically by dipping the teat into the composition. By “treat or protect,” it is meant that the composition once applied maintains or improves the health or condition of the teat, specifically the health or condition of the skin of the teat. Thus, for instance, the composition can treat or protect against infection, such as mastitis, or treat or protect against drying, chapping, or cracking of the skin of the teat.

The following examples further illustrate the invention but, of course, should not be construed as in any way limiting its scope.

### EXAMPLE 1

The following Example illustrates that isopropyl alcohol, citric acid, and parabens tolerate gamma radiation sterilization, but methylene blue degrades upon gamma irradiation.

An antimicrobial composition comprising isopropyl alcohol, citric acid, methyl paraben, propyl paraben, and methylene blue in HDPE bottles was exposed to 25-40 kGy radiation, and the amount of each component detected in the solution before and after radiation was tested. The testing was repeated after 5 months of storage. The results are presented in Tables 1 and 2, below, wherein “exposed” indicates the samples exposed to gamma radiation and “unexposed” indicates the samples that were not exposed to gamma radiation.

As shown in the tables, the change in IPA, citric acid, and parabens was negligible, indicating that gamma radiation did not degrade these components. However, the gamma radiation degraded approximately 50% of the methylene blue in solution, which was accompanied by a color change.

TABLE 1
HDPE Bottle Concentration % Change, T = 0 vs. T = 5 Months

| | Total Citric Acid % LC | | % Change |
|---|---|---|---|
| Sample | Unexposed | Exposed | (Unexposed vs. Exposed) |
| HDPE | 96.3 | 97.4 | 1.1 |
| HDPE | 93.1 | 93.5 | 0.4 |
| T = 5 months | | | |
| % Change | −3.3 | −4.0 | |
| | % IPA | | % Change |
| Sample | Unexposed | Exposed | (Unexposed vs. Exposed) |
| HDPE | 68.1 | 68.6 | 1.2 |
| HDPE | 69.2 | 69.1 | −0.1 |
| T = 5 months | | | |
| % Change | 1.6 | 0.7 | |
| | Methylparaben % LC | | % Change |
| Sample | Unexposed | Exposed | (Unexposed vs. Exposed) |
| HDPE | 74.8 | 74.5 | −0.4 |
| HDPE | 75.2 | 75.0 | −0.3 |
| T = 5 months | | | |
| % Change | 0.5 | 0.7 | |
| | Propylparaben % LC | | % Change |
| Sample | Unexposed | Exposed | (Unexposed vs. Exposed) |
| HDPE | 74.9 | 74.9 | 0 |
| HDPE | 75.3 | 75.2 | −0.1 |
| T = 5 months | | | |
| % Change | 0.5 | 0.4 | |

TABLE 2
Methylene Blue Total Impurity % Change T = 0 vs. T = 5
Months (650 nm)

| | Total Methylene | | % Change |
|---|---|---|---|
| | Blue % LC | | (Unexposed vs. |
| Sample | Unexposed | Exposed | Exposed) |
| HDPE T = 0 | 105.5 | 44.9 | −57.4 |
| HDPE T = 5 | 93.4 | 47.3 | −49.4 |
| months | | | |

### EXAMPLE 2

The following example demonstrates a synergistic antimicrobial effect between alcohol and parabens in a composition that is suitable for gamma sterilization and has low-alcohol levels.

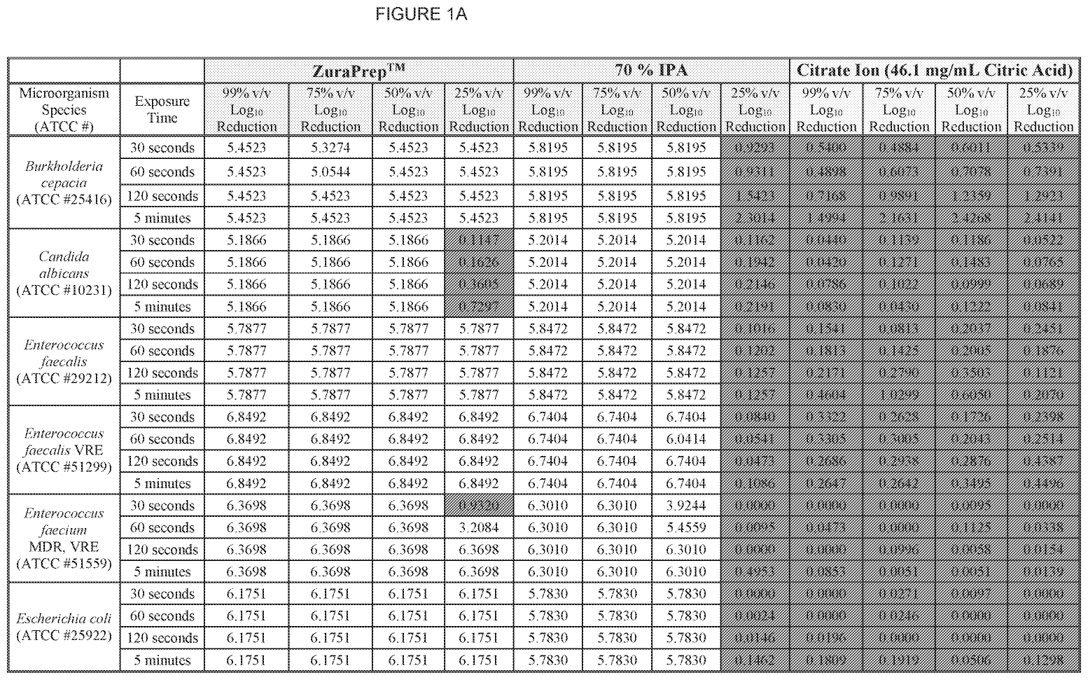

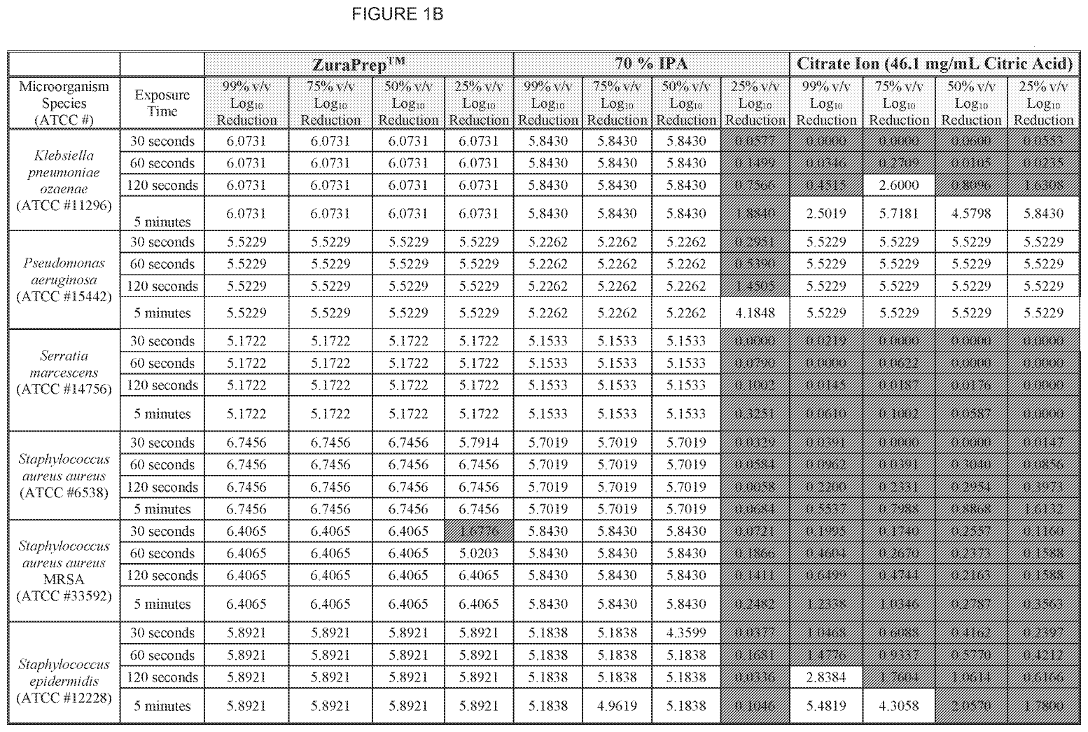

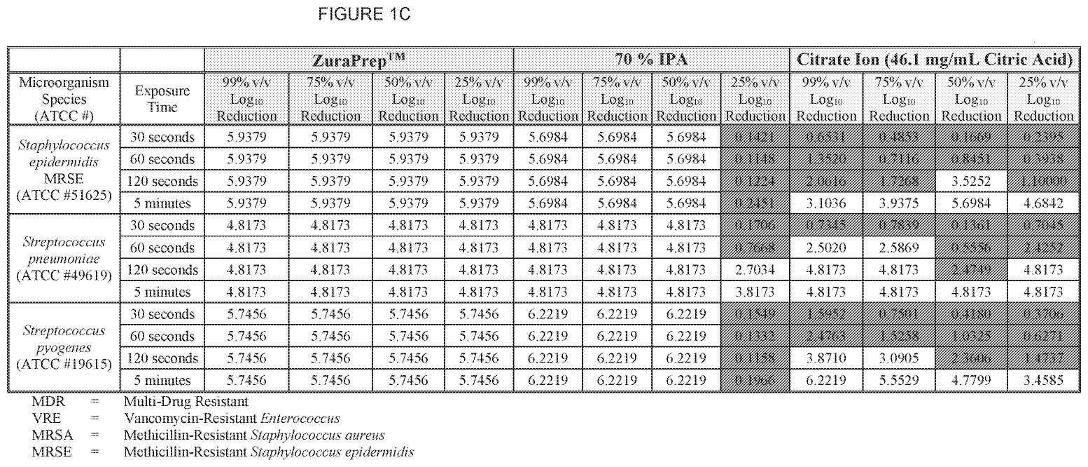

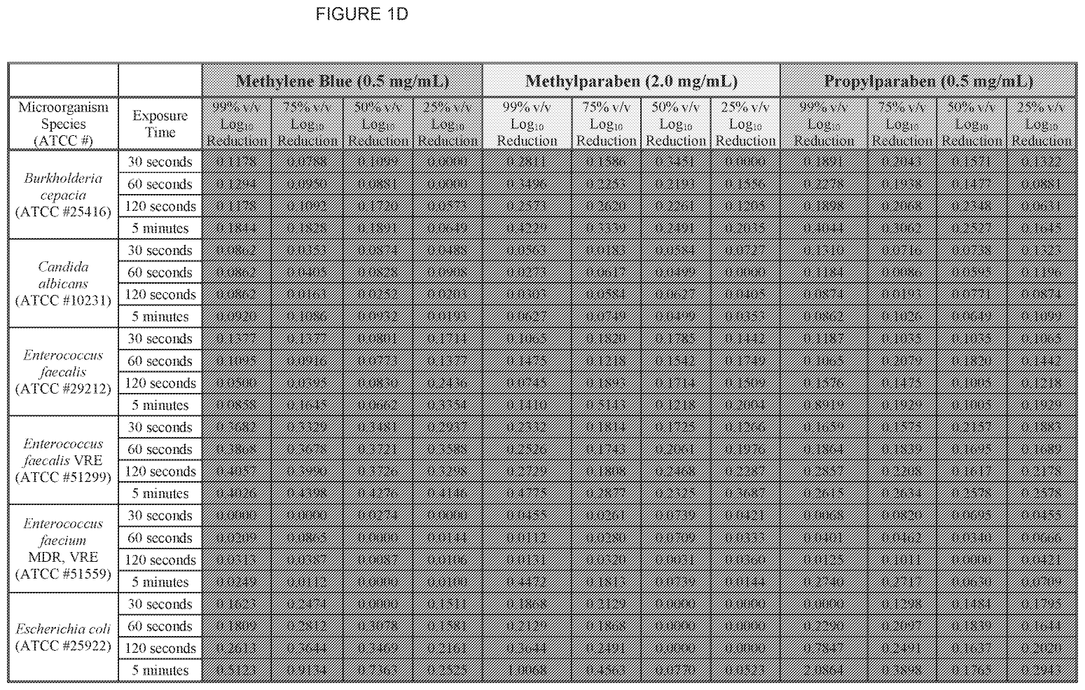

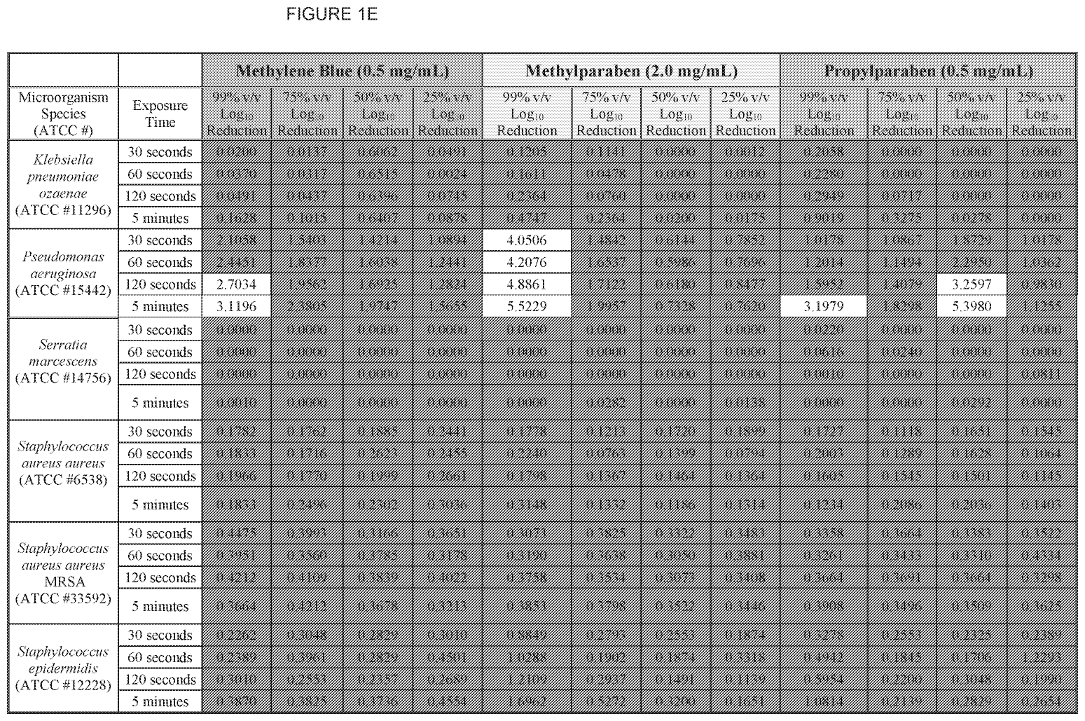

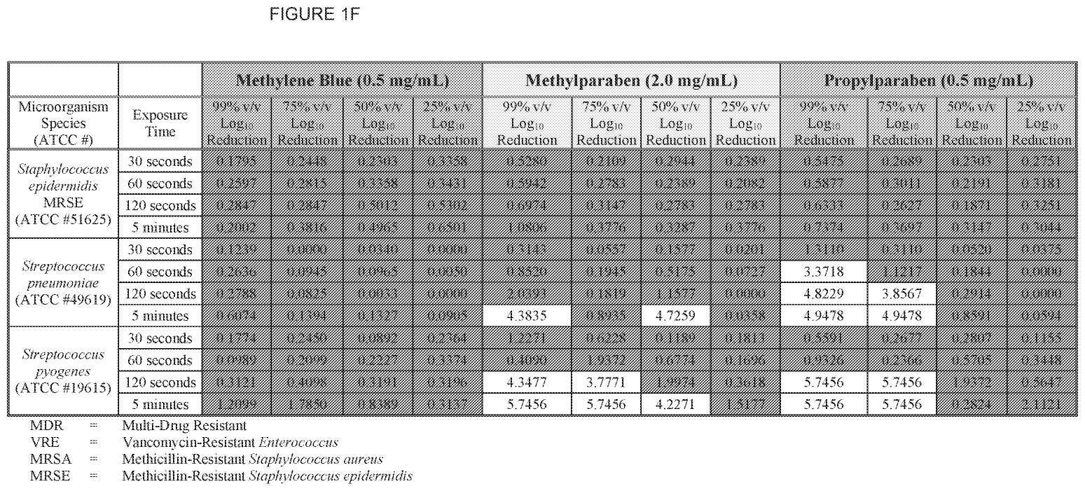

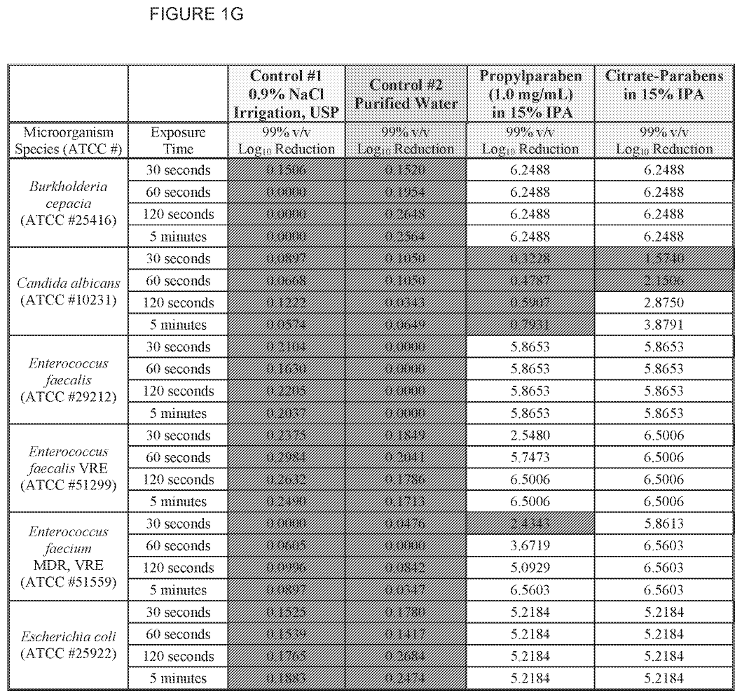

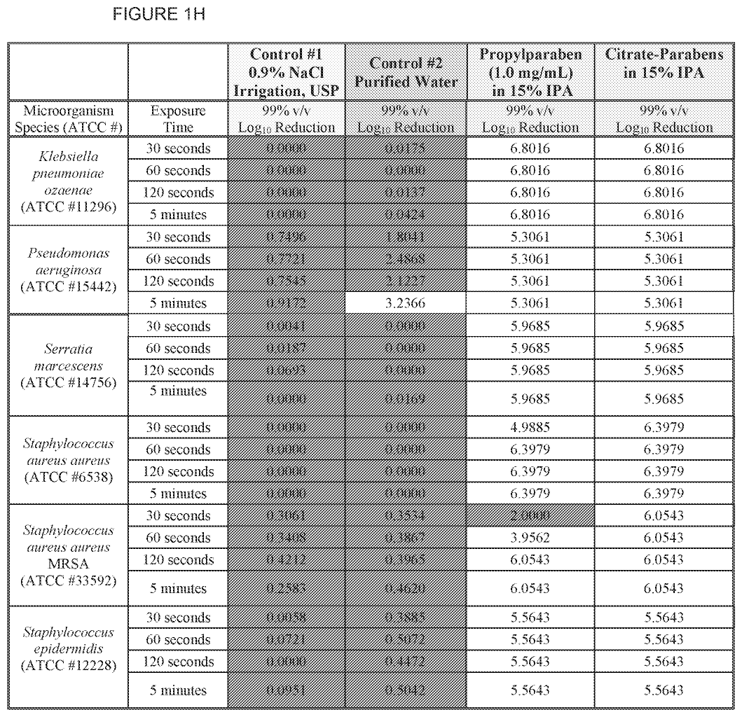

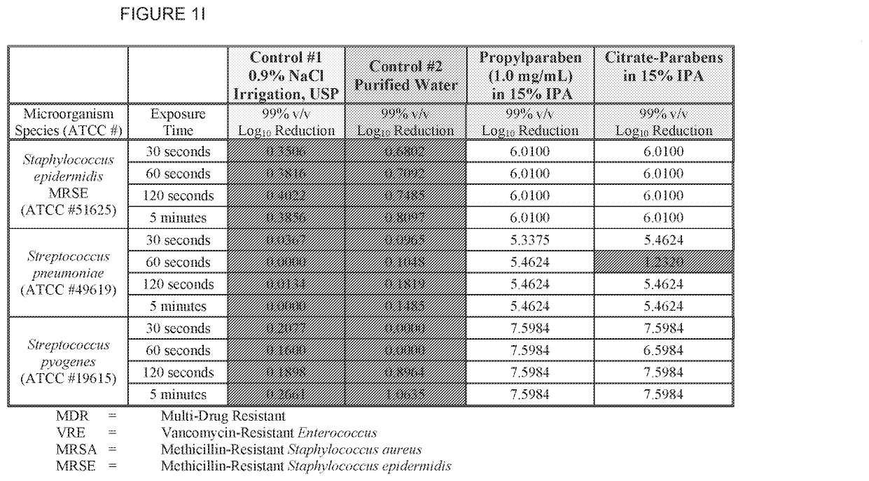

The antimicrobial effect of various components of the composition of Example 1 were tested individually and together at various dilutions. The results are presented in FIG. 1A-1I, wherein shaded boxes indicate no significant response (<3 log10 reduction).

As indicated in the table, IPA alone showed little to no effect at the 25% v/v strength solution against most tested microorganisms. Similarly, parabens, methylene blue, and citrate alone showed little to no effect at all tested concentrations against most tested microorganisms.

However, when combined with only 15% IPA, the parabens with or without citrate showed remarkable antimicrobial effect against most microorganisms tested (FIGS. 1G-1I).

### EXAMPLE 3

The following Example illustrates the preparation and testing of a low-alcohol antimicrobial composition comprising isopropyl alcohol, citric acid, and parabens.

### Xanthan Gum Solution

3.5 g of Xanthan Gum was added to 696.5 g warm DI water (˜60-75° F.). The composition was mixed using an overhead mixer with a cowles type blade on a shaft, at medium speed, until homogeneous solution was achieved (about 20 minutes) to provide a 0.05 wt. % Xantham gum solution.

Alcohol and Paraben solutions with 3% and 4% (w/w) IPA

Propylene glycol was added to methylparaben and propylparaben in the amounts shown in Table 3, with no mixing [symbol] Then, citric acid, urea, and trisodium citrate dihydrate in required amounts to the propylene glycol/paraben mixture. The mixture was stirred until a homogeneous solution was obtained, i.e. no precipitation. The indicated amount of IPA was added to the solution while continuously mixing. While still mixing, the required weight of the 0.5% (w/w) Xanthan Gum/DI Water solution (prepared above) was added to the formulation. Mixing was continued until the solution was homogeneous, about 5 minutes. All the solid components were completely dissolved in the solution. While continuously mixing, the pH of the solution was adjusted by adding 10N sodium hydroxide dropwise until the target pH (3.5-4.0) was reached.

TABLE 3

| | | | | Formulation | |
|---|---|---|---|---|---|
| | | | | (weight, g) | |
| | | Target | | 4% w/w | 3% w/w |
| Component | | (w/w) % | (g) | IPA | IPA |
| Isopropyl Alcohol | | 4.0 or 3.0% | 20.0 or 15.0 | 20.000 | 15.000 |
| Urea | | 2.0% | 10.0 | 10.0035 | 10.0604 |
| Propylene Glycol | | 20.0% | 100.0 | 100.000 | 100.0228 |
| Anhydrous citric | | 4.35% | 21.75 | 21.7782 | 21.7675 |
| acid | | | | | |
| Trisodium citrate | | 0.40% | 2.0 | 2.0606 | 2.0154 |
| dihydrate | | | | | |
| Methylparaben | | 0.40% | 2.0 | 2.0114 | 2.0162 |
| Propylparaben | | 0.17% | 0.85 | 0.8522 | 0.8546 |
| Xanthan Gum | | 0.50% | 2.5 (XG), | 335.56 ¹ | 340.1 ¹ |
| (XG) Solution¹ | | (XG), | 332 or 337 | | |
| | | 66.4% | (H₂0) | | |
| | | or | | | |
| | | 67.4% | | | |
| | | (H₂0) | | | |
| 10N NaOH | | ~1.78 | ~8.9 | 8.96 | 9.0 |
| Results | Total | 100.0% | 500.0 | 501.23 | 500.8 |
| | Weight | | | | |

| | | | |
|---|---|---|---|
| pH | 3.5-4.0 | 3.51 | 3.52 |
| Density | 1.12-1.14 | 1.140 | 1.140 |
| (g/mL) | | | |

¹ Xanthan gum solution (in water) at a final concentration of 0.50% was used in the formulations. Xanthan gum concentrations in final formulations were <0.50% w/w (4% w/w IPA = 0.33% w/w Xanthan Gum (1.67 g); 3% w/w IPA = 0.34% w/w Xanthan Gum (1.7 g)

[symbol]

### Flash Point Test Results

An Elcometer 6910/1 SETA flash ‘Series 3’ Closed Cup Tester (FIG. 1) with automatic flash detection mechanism was used to measure the flash point of the solutions according to the procedure of ASTM D3278, which closely follows the consumer products safety commissions method 16 CFR 1500. ASTM D3278 is also similar to ASTM D93 which is meant for larger volume samples. The 4% alcohol solution had a flash point of 58° C., and the 3% alcohol solution had a flash point of 64° C.

Additional samples of the 4 wt. % IPA and 3 wt. % IPA formulations were prepared with Xanthan Gum at a concentration of 0.5% (w/w) in the final formulation. The flash points of these repeat formulations, using the SETA closed cup, were 60° C. for 4 wt. % IPA and 65° C. for 3 wt. % IPA.

### MicroChem Kill Study Results

Suspension time kill studies were performed according to ASTM E2315, Assessment of Antimicrobial Activity using a Time-Kill Procedure, which is a quantitative test method designed to assess changes in the population of organisms in an antimicrobial liquid suspension. The 4 wt. % IPA and 3 wt. % IPA formulations were tested against Candida h using contact times of 1, 2, and 5 minutes. The results are provided in Table 4.

Both the 3% IPA and 4% IPA formulations yielded >3 average log10 reductions of the CFU/mL (compared to the control at time zero) at all three time points tested (1, 2, and 5 minutes).

TABLE 4
C. albicans time-kill study results

| | | | | | | Percent | Log₁₀ |
|---|---|---|---|---|---|---|---|
| | | | | | | Reduction | Reduction |
| | | Contact | | | | Compared | Compared |
| Test | Test | Time | | | Average | to Control | to Control |
| Microorganism | Substance | (min) | Replicate | CFU/ml | CFU/ml | at Time Zero | at Time Zero |
| C. albicans | Numbers | Time | 1 | 1.05E+07 | 1.07E+07 | N/A | |
| ATCC 10231 | Control | Zero | 2 | 1.04E+07 | | | |
| | | | 3 | 1.13E+07 | | | |
| | TK-1 | 1 | 1 | 3.26E+03 | 3.94E+03 | 99.96% | 3.43 |
| | 3% IPA | | 2 | 4.80E−03 | | | |
| | | | 3 | 3.75E−03 | | | |
| | | 2 | 1 | 2.91E−03 | 2.89E+03 | 99.97% | 3.57 |
| | | | 2 | 3.37E−03 | | | |
| | | | 3 | 2.40E−03 | | | |
| | | 5 | 1 | 2.05E+03 | 2.12E+03 | 99.98% | 3.70 |
| | | | 2 | 2.17E+03 | | | |
| | | | 3 | 2.14E+03 | | | |
| | TK-1 | 1 | 1 | 5.20E+03 | 6.66E+03 | 99.94% | 3.21 |
| | 4% IPA | | 2 | 6.95E+03 | | | |
| | | | 3 | 7.83E+03 | | | |
| | | 2 | 1 | 4.81E−03 | 4.18E+03 | 99.96% | 3.41 |
| | | | 2 | 3.89E−03 | | | |
| | | | 3 | 3.84E+03 | | | |
| | | 5 | 1 | 2.87E+03 | 3.03E+03 | 99.97% | 3.55 |
| | | | 2 | 3.06E−03 | | | |
| | | | 3 | 3.17E−03 | | | |

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An antimicrobial composition comprising (a) about 18% or less alcohol and (b) a paraben.

2. (canceled)

3. The composition of claim 1, wherein the active ingredient consists of (a) alcohol, (b) a paraben, and optionally (c) citric acid or citrate.

4. (canceled)

5. The composition of claim 1, wherein the composition consists essentially of:
(i) an active ingredient that consists of (a) about 5 wt. % or less alcohol, (b) a paraben, and optionally (c) citric acid or citrate,
(ii) optionally one or more of a thickener, emollient, surfactant, or urea, and
(iii) water;
or wherein the composition consists of:
(i) an active ingredient that consists of (a) about 5 wt. % or less alcohol, (b) a paraben, and optionally (c) citric acid or citrate,
(ii) optionally one or more of a thickener, emollient, surfactant, or urea, and
(iii) water.

6. (canceled)

7. The composition of claim 1, wherein the composition is a hand disinfectant and comprises glycerin, glycol, acrylates, acrylate crosspolymers, aloe, or lanolin.

8. A sterile or sterilizable antimicrobial composition comprising (a) alcohol and (b) paraben, and is substantially or completely free of any component that degrades upon application of a sterilizing dose of gamma radiation, or upon application of at least 25 KGy of gamma radiation.

9. (canceled)

10. (canceled)

11. The composition of claim 8, wherein the active ingredient consists of (a) alcohol, (b) a paraben, and optionally (c) citric acid or citrate.

12. The composition of claim 8, wherein the composition consists essentially of (a) alcohol, (b) paraben, (c) water, optionally (d) citrate, and optionally (e) a food-grade colorant
or wherein the composition consists of (a) alcohol, (b) paraben, (c) water, optionally (d) citrate, and optionally (e) a food-grade colorant.

13. (canceled)

14. The composition of claim 8, wherein the composition comprises 20% or less alcohol.

15. (canceled)

16. (canceled)

17. The composition of claim 8, wherein the composition comprises about 2-4% alcohol.

18. (canceled)

19. The composition of claim 8, wherein the alcohol is ethanol, isopropyl alcohol, n-propanol, or mixture thereof

20. The composition of claim 8, wherein the composition has a flash point of 50° C. or greater when tested under ASTM D3278.

21. (canceled)

22. The composition of claim 8, wherein the composition produces a log-kill of Candida albicans of 3 or greater after contact for 1 minute using ASTM E2315.

23. (canceled)

24. The composition of claim 8, wherein the composition comprises up to the solubility limit of paraben, optionally 0.1-3 wt % paraben.

25. (canceled)

26. The composition of claim 8, wherein the composition comprises about 1% to about 10% by weight citric acid.

27. The composition of claim 8, wherein the composition comprises about 0.2% to about 1% by weight sodium citrate.

28. (canceled)

29. The composition of claim 8, wherein the composition is substantially free of methylene blue, chlorhexidine, or iodine.

30. The composition of claim 8, wherein the composition is contained within an applicator comprising an absorbent material optionally comprising a handle portion comprising a chamber for housing the antimicrobial composition, and an applicator portion comprising an absorbent material in fluid communication with the handle portion; or wherein the composition is absorbed into a fiber cloth or wipe; or wherein the composition is in a sealed, single-use container that does not degrade upon application of 25 KGy gamma radiation.

31. (canceled)

32. (canceled)

33. (canceled)

34. A fiber cloth or wipe comprising the composition of claim 8 sealed in a single-use package, wherein the package and cloth or wipe are sterilized or sterilizable.

35. A method of disinfecting a skin surface comprising applying the composition of any of claim 8 to the skin surface, optionally wherein the skin surface is an open soft tissue wound, a surgical site, or the skin of a person's hands.

36.-39. (canceled)

40. A method of preparing a sterile antimicrobial composition comprising
(i) providing a composition comprising (a) alcohol, (b) paraben, (c) water, and optionally citric acid or citrate, and
(ii) applying about 15 kGy or more gamma radiation, optionally about 25 KGy or more gamma radiation, to the composition to provide a sterile antimicrobial composition.

41.-43. (canceled)
